(19) United States
(12) Patent Application Publication
(10) Pub. No.: US 2023/0159910 A1
(43) Pub. Date: May 25, 2023

(54) DIMETHYLMONOTHIOARSINIC ACID-INDUCED MALIGNANTLY TRANSFORMED CELL LINE OF HUMAN KERATINOCYTES AND USE THEREOF

(71) Applicant: SOOCHOW UNIVERSITY, Suzhou (CN)

(72) Inventors: Yan AN, Suzhou (CN); Rui YAN, Suzhou (CN); Jiayuan MAO, Suzhou (CN); Qianlei YANG, Suzhou (CN); Haixuan XIA, Suzhou (CN); Qi KONG, Suzhou (CN); Yuanjie WEI, Suzhou (CN)

(21) Appl. No.: 17/631,478

(22) PCT Filed: Feb. 20, 2021
(86) PCT No.: PCT/CN2021/077088

§ 371 Date: Jan. 30, 2022

(30) Foreign Application Priority Data
Jan. 25, 2021 (CN) 202110109540.3

(51) Int. Cl. C12N 15/01; C12N 5/09; G01N 33/50

(52) U.S. Cl. CPC C12N 15/01; C12N 5/0693; G01N 33/5017

(57) ABSTRACT

The present invention relates to the technical field of model establishment, and provides a dimethylmonothioarsinic acid-induced malignantly transformed cell line of human keratinocytes and use thereof. In the present invention, human keratinocytes are persistently exposed to and incubated with dimethylmonothioarsinic acid, to construct an inorganic arsenic metabolite dimethylmonothioarsinic acid (DMMTA^{v})-induced malignantly transformed cell model of human keratinocytes. The malignantly transformed cell model of the present invention promotes the identification of carcinogenicity of arsenic methylated metabolites, and indicates that long-term exposure to low-dose arsenic metabolite dimethylmonothioarsinic acid (DMMTA^{v}) causes malignant transformation of skin cells, thus providing a new cell model basis and new research idea for the study of carcinogenic mechanism of arsenic.

### FIELD OF THE INVENTION

The present invention relates to the technical field of model establishment, and more particularly to a dimethylmonothioarsinic acid-induced malignantly transformed cell line of human keratinocytes and usethereof.

### DESCRIPTION OF THE RELATED ART

Inorganic arsenic and its compounds are confirmed carcinogens for humans. Previously, it is often considered that the metabolism of inorganic arsenic in the body is a process of detoxification. However, recent studies have found that it produces highly toxic product dimethylmonothioarsinic acid (DMMTA^{v}) during the metabolic process, which may be closely related to the occurrence of cancer. Due to the lack of relevant animal models, the specific molecular mechanism of carcinogenesis is still unclear, resulting in a lack of clinically effective early diagnosis and treatment methods.

At present, the research on the carcinogenic mechanism of arsenic mainly involves the use of an in-vitro malignantly transformed cell model established by chronic exposure to inorganic arsenic, and the malignantly transformed cell model is a good material for studying the carcinogenic mechanism of carcinogens. However, the in-vitro malignantly transformed cell model established by chronic exposure to inorganic arsenic has its limitations, in which the metabolic transformation of arsenic and the high toxicity of metabolites in the body are not fully considered. In addition, because the genome of human cells is relatively stable and has an effective DNA repair mechanism, it has potent resistance to external damage, and is especially less sensitive to damages caused by carcinogens than some animal cells, and is more difficult to transform compared to animal cells. There is no report on the malignantly transformed cell model induced by dimethylmonothioarsinic acid.

### SUMMARY OF THE INVENTION

To solve the above problems, an inorganic arsenic metabolite dimethylmonothioarsinic acid-induced malignantly transformed cell model of human keratinocytes is constructed in the present invention, which promotes the identification of carcinogenicity of arsenic methylated metabolites, and provides a new idea and model basis for the study of carcinogenic mechanism of arsenic.

A first object of the present invention is to provide a dimethylmonothioarsinic acid-induced malignantly transformed cell line of human keratinocytes, which is deposited in China General Microbiological Culture Collection Center (CGMCC, Address: Building #3, NO.1 Beichen West Road, Chaoyang District, Beijing) under CGMCC Accession No. 21419 on Dec. 30, 2020.

A second object of the present invention is to provide a method for constructing a dimethylmonothioarsinic acid-induced malignantly transformed cell line of human keratinocytes. The method includes specifically persistently exposing and incubating human keratinocytes in a medium containing 0.5-1.0 μM dimethylmonothioarsinic acid, refreshing the medium every 20-30 h, and sub-culturing and expanding to the 30-40th passages when the cells has a confluency reaching 75-85%, to obtain the dimethylmonothioarsinic acid-induced malignantly transformed cell line of human keratinocytes.

Preferably, the dimethylmonothioarsinic acid is prepared by a method including:

S1. dissolving dimethylarsonic acid and sodium sulfate in water, and slowly adding concentrated sulfuric acid into the resulting solution and mixing well by stirring for 10-24 h, wherein a molar ratio of dimethylarsonic acid, sodium sulfate and concentrated sulfuric acid is 1:1.5-2:1.5-2;

S2. adding hydrochloric acid into the mixed solution obtained in S1 to perform a dehydration reaction, then extracting with chloroform after the dehydration reaction to obtain an organic phase;

S3. adding a saturated salt water to the organic phase obtained in S2, to collect the subnatant;

S4. adding anhydrous calcium chloride to the subnatant to collect the supernatant, heating to remove the water to obtain a solid; and

S5. recrystallizing the solid obtained in S4 in hexane, and removing hexane to obtain dimethylmonothioarsinic acid.

Preferably, the recrystallization inludes standing at 0-4° C. for 10-15 h. Preferably, the medium is Dulbecco's modified Eagle's medium (DMEM) with high glucose.

Preferably, the Dulbecco's modified Eagle's medium (DMEM) with high glucose includes 80-120 μg/ml streptomycin, 80-120 U/ml penicillin and 8-12% fetal bovine serum.

Preferably, the method further comprises indicating the malignant transformation of cells by detecting the secretion of MMP-9, the migration ability or the soft agar colony formation ability of the cells.

A third object of the present invention is to provide use of the dimethylmonothioarsinic acid-induced malignantly transformed cell line of human keratinocytes as a cell model in the study of carcinogenic mechanism of dimethylmonothioarsinic acid.

A fourth object of the present invention is to provide use of the dimethylmonothioarsinic acid-induced malignantly transformed cell line of human keratinocytes in screening or evaluating drugs for the treatment of cancers caused by inorganic arsenic.

The present invention has the following beneficial effects.

In the present invention, an inorganic arsenic metabolite dimethylmonothioarsinic acid (DMMTA^{v})-induced malignantly transformed cell model of human keratinocytes is constructed. The malignantly transformed cell model promotes the identification of carcinogenicity of arsenic methylated metabolites, and indicates that long-term exposure to low-dose arsenic metabolite dimethylmonothioarsinic acid (DMMTA^{v}) causes malignant transformation of skin cells, thus providing a new cell model basis and new research idea for the study of carcinogenic mechanism of arsenic.

Deposit of Biological Material:

dimethylmonothioarsinic acid-induced malignantly transformed cell line of human keratinocytes, deposited in China General Microbiological Culture Collection Center (CGMCC, Address: Building #3, NO.1 Beichen West Road, Chaoyang District, Beijing) under CGMCC Accession No. 19650 on Jun. 3, 2020.

### BRIEF DESCRIPTION OF THE DRAWINGS

FIG. 1 shows the cell survival of HaCaT cells 24 h after treatment with various concentrations (0-2 μM) of DMMTA^{v}.

FIG. 2 shows the changes in MMP-9 secretion after persistent exposure of the HaCaT cells sub-cultured to different passages to 1.0 μM DMMTA^{v}. Note: *: compared with the passage control group (without treatment), P<0.05.

FIG. 3 shows the changes in cell doubling time after persistent exposure of the HaCaT cells sub-cultured to different passages to 1.0 μM DMMTA^{v}. Note: *: compared with the passage control group (without treatment), P<0.05.

FIG. 4 shows the changes in cell migration ability after persistent exposure of the HaCaT cells sub-cultured to the 35th passage to 1.0 μM DMMTA^{v}. Note: The left panel shows the images of the cell scratch assay of the 35th passage in the exposure group and the passage control group observed under a microscope; and the right panel shows the quantitative analysis of the left panel. Note: *: compared with the passage control group (without treatment), P<0.05.

FIG. 5 shows the changes in colony forming ability of cells after persistent exposure of the HaCaT cells sub-cultured to the 35th passage to 1.0 μM DMMTA^{v}. Note: The upper panel shows the images of the soft agar colony forming assay of the 35th passage in the exposure group and the passage control group observed under a microscope, and the lower panel shows the quantitative analysis of the upper panel. Note: *: compared with the passage control group (without treatment), P<0.05.

### DETAILED DESCRIPTION OF THE PREFERRED EMBODIMENTS

The present invention will be further described below with reference to the accompanying drawings and specific examples, so that those skilled in the art can better understand and implement the present invention; however, the present invention is not limited thereto.

### EXAMPLE 1

Synthesis of inorganic arsenic metabolite dimethylmonothioarsinic acid (DMMTA^{v}): 5 g (about 35.6 mmol) of DMA^{v} powder (molecular weight 138) and 4.5 g (about 57.0 mmol) of sodium sulfide (Na₂S) powder were weighed and dissolved in 150 mL ultrapure water. Then concentrated sulfuric acid was slowly added to the resulting mixture (the final molar ratio H₂SO₄:Na₂S: DMAV=1.6:1.6:1), and then the mixture was stirred overnight with a magnetic stirrer under an argon atmosphere. The next day, hydrochloric acid was added to the mixture so that DMMTA^{v} suffers from dehydration reaction, then chloroform was added for extraction. Subsequently saturated salt water was added and the subnatant was collected in a beaker. anhydrous calcium chloride was added to the collected subnatant and then the supernatant was collected, and the water was removed by heating in water bath. Hexane and trace mount of DMMTA^{v} solid were added for recrystallization. The mixture was placed overnight in a refrigerator. Next day, the mixture was taken from the refrigerator and hexane was added, finally a white solid was obtained after evaporation and drying under argon atmosphere. The obtained white solid was confirmed by high performance liquid chromatography-inductively coupled plasma mass spectrometry (HPLC-ICP-MS).

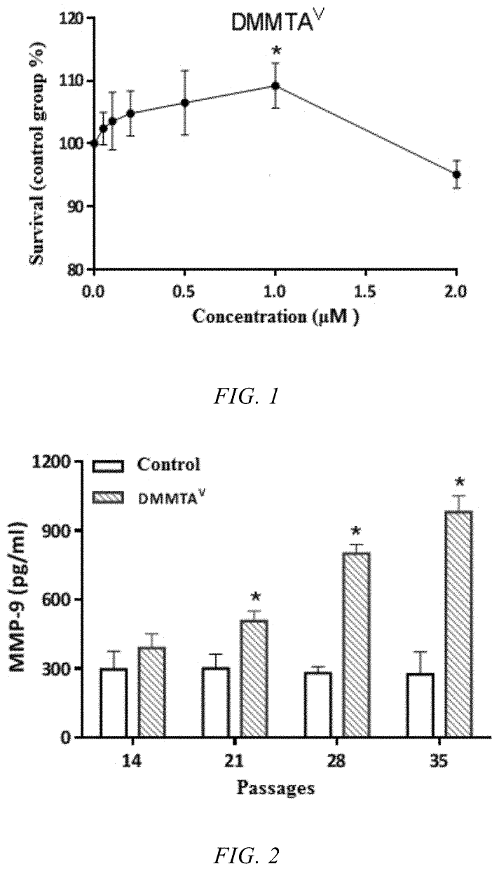

Determination of appropriate exposure dose: After the HaCaT cells were treated with various concentrations (0-2 μM) of DMMTA^{v} for 24 h, and the cell survival was detected by the CCK-8 kit. The dose with the highest cell survival rate was used as the dose for chronic exposure, as shown in FIG. 1. The chronic exposure dose of DMMTA^{v} is determined to be 1.0 μM.

### EXAMPLE 2

Two dishes of normal HaCaT cells (cell passage 0) were prepared. One dish of cells was persistently and chronically exposed to and incubated with Dulbecco's modified Eagle's medium (DMEM) with high glucose with 1.0 μM DMMTAv (containing 100 μg/ml streptomycin, 100 U/ml penicillin and 10% fetal bovine serum), and the medium was refreshed every 24 h. When the cells were grown to a confluency reaching 80%, the cells were sub-cultured and expanded up to the 35th passage. The other dish of cells was persistently and chronically exposed to and normally incubated with Dulbecco's modified Eagle's medium (DMEM) with high glucose (containing 100 μg/ml streptomycin, 100 U/ml penicillin and 10% fetal bovine serum), and the medium was refreshed every 24 h. When the cells were grown to a confluency reaching 80%, the cells were normally sub-cultured and expanded up to the 35th passage and used as the passage control group. One monitoring point is set every 7 passages to detect the cell doubling time of the 0th, 1st, 7th, 14th, 21st, 28th, 35th passages in the exposure group and the passage control group.

### EXAMPLE 3

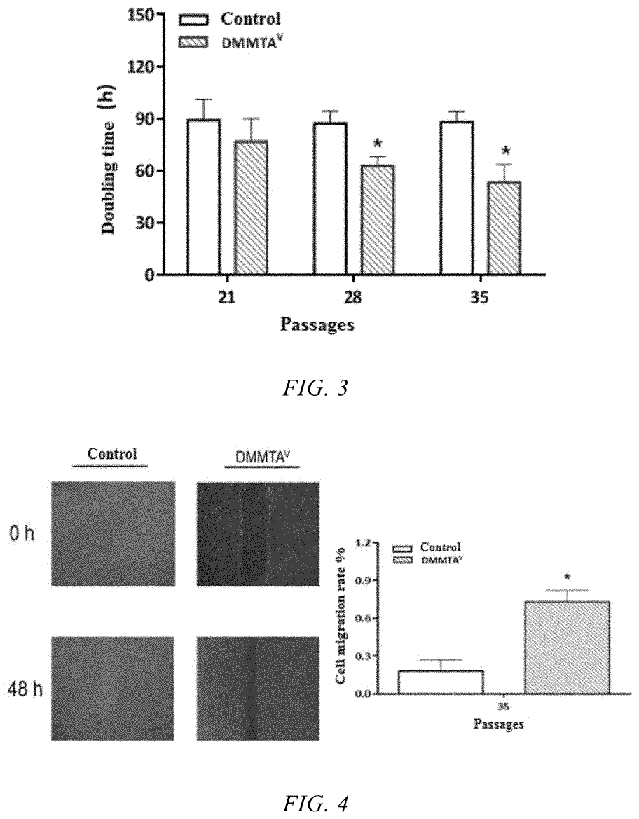

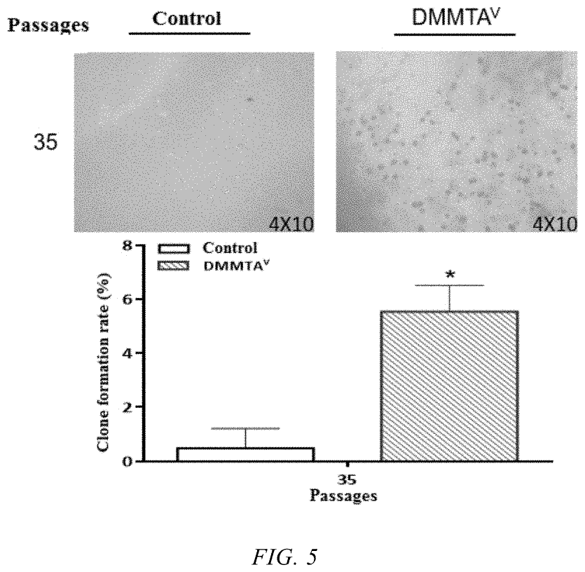

By detecting the changes in the matrix metalloproteinase-9 (MMP-9), the cell doubling time, the cell migration ability and cell anchoring independent growth, whether the cell undergoes malignant transformation is determined. FIG. 2 reflects the changes in MMP-9 secretion in HaCaT cells of various passages. It can be seen that the secretion of MMP-9 in the 21st, 28th and 35th passages incubated by persistently exposing to dimethylmonothioarsinic acid is significantly increased compared with the passage control group. FIG. 3 reflects the changes in doubling time of HaCaT cells of various passages. It can be seen that the cell doubling time of the 28th, 35th passage incubated by persistently exposing to dimethylmonothioarsinic acid is significantly reduced. FIG. 4 shows the changes in cell migration ability of HaCaT cells of the 35th passage detected by scratch test. It can be seen that the cell migration ability of the 35th passage incubated by persistently exposing to dimethylmonothioarsinic acid is significantly enhanced. FIG. 5 shows the changes in cell anchoring independent growth detected by soft agar colony forming test of the 35th passage incubated by exposing to dimethylmonothioarsinic acid. It can be seen that the cells in the passage control group only form a few tiny colonies in soft agar, and the cells of the 35th passage incubated by persistently exposing to dimethylmonothioarsinic acid can form obvious colonies in soft agar, with a colony formation rate significantly higher than that of the passage control group cells. The MMP-9 secretion, the cell doubling time, the cell migration ability and the soft agar colony forming ability mentioned above are all commonly used indicators to identify malignant transformation of cells in vitro. According to the above experimental results, subculture to 35th passage by long-term exposure to 1.0 μM dimethylmonothioarsinic acid (DMMTA^{v}) can induce HaCaT cells to undergo malignant transformation.

The above-described embodiments are merely preferred embodiments for the purpose of fully illustrating the present invention, and the scope of the present invention is not limited thereto. Equivalent substitutions or modifications can be made by those skilled in the art based on the present invention, which are within the scope of the present invention as defined by the claims.

What is claimed is:

1. A dimethylmonothioarsinic acid-induced malignantly transformed cell line of human keratinocytes, which is deposited in China General Microbiological Culture Collection Center (CGMCC, Address: Building #3, NO.1 Beichen West Road, Chaoyang District, Beijing) under CGMCC Accession No. 21419 on Dec. 30, 2020.

2. A method for constructing a dimethylmonothioarsinic acid-induced malignantly transformed cell line of human keratinocytes according to claim 1, comprising persistently exposing and incubating human keratinocytes in a medium containing 0.5-1.0 μM dimethylmonothioarsinic acid, refreshing the medium every 20-30 h, and sub-culturing and expanding to the 30-40 th passages when the cells has a confluency reaching 75-85%, to obtain the dimethylmonothioarsinic acid-induced malignantly transformed cell line of human keratinocytes.

3. The method according to claim 2, wherein the dimethylmonothioarsinic acid is prepared by a method comprising:
S1. dissolving dimethylarsonic acid and sodium sulfate in water, and slowly adding concentrated sulfuric acid into the resulting solution and mixing well by stirring for 10-24 h, wherein a molar ratio of dimethylarsonic acid, sodium sulfate and concentrated sulfuric acid is 1:1.5-2:1.5-2;
S2. adding hydrochloric acid into the mixed solution obtained in S1 to perform a dehydration reaction, then extracting with chloroform after the dehydration reaction to obtain an organic phase;
S3. adding a saturated salt water to the organic phase obtained in S2, to collect the subnatant;
S4. adding anhydrous calcium chloride to the subnatant to collect the supernatant, heating to remove the water to obtain a solid; and
S5. recrystallizing the solid obtained in S4 in hexane, and removing hexane to obtain dimethylmonothioarsinic acid.

4. The method according to claim 3, wherein the recrystallization comprises standing at 0-4° C. for 10-15 h.

5. The method according to claim 2, wherein the medium is Dulbecco's modified Eagle's medium with high glucose.

6. The method according to claim 5, wherein the Dulbecco's modified Eagle's medium with high glucose comprises 80-120 μg/ml streptomycin, 80-120 U/ml penicillin and 8-12% fetal bovine serum.

7. The method according to claim 2, wherein the method further comprises indicating the malignant transformation of cells by detecting the secretion of MMP-9, the migration ability or the soft agar colony formation ability of the cells.

8. Use of the dimethylmonothioarsinic acid-induced malignantly transformed cell line of human keratinocytes according to claim 1, as a cell model in the study of carcinogenic mechanism of dimethylmonothioarsinic acid.

9. Use of the dimethylmonothioarsinic acid-induced malignantly transformed cell line of human keratinocytes according to claim 1 in screening or evaluating drugs for the treatment of cancers caused by inorganic arsenic.
